(19) United States
(12) Patent Application Publication
(10) Pub. No.: US 2016/0155953 A1
(43) Pub. Date: Jun. 2, 2016

(54) MATERIAL FOR ORGANIC ELECTROLUMINESCENT DEVICE AND ORGANIC ELECTROLUMINESCENT DEVICE USING THE SAME

(71) Applicant: SAMSUNG DISPLAY CO., LTD., Yongin-si (KR)

(72) Inventors: Hisayuki Kawamura, Yokohama (JP); Seokhwan Hwang, Suwon-si (KR); Hiroaki Itoi, Yokohama (JP); Masatsugu Ueno, Yokohama (JP); Xiulan Jin, Yokohama (JP); Naoya Sakamoto, Yokohama (JP); Junta Fuchiwaki, Yokohama (JP); Hideo Miyake, Yokohama (JP)

(21) Appl. No.: 14/925,965

(22) Filed: Oct. 28, 2015

(30) Foreign Application Priority Data
Dec. 1, 2014 (JP) 2014-242824

(51) Int. Cl. H01L 51/00; C07D 209/86; C07D 333/76; C07D 307/91; C09K 11/02

(52) U.S. Cl. CPC H01L 51/0061; C07D 307/91; C09K 11/025; C07D 333/76; H01L 51/006; C07D 209/86; H01L 51/5012

(57) ABSTRACT

A material for an organic electroluminescent device is represented by the following Formula 1:

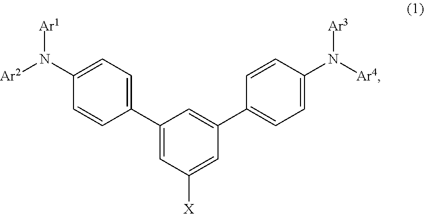

where Ar₁ to Ar₄ may be each independently selected from a substituted or unsubstituted aryl group having 6 to 30 carbon atoms for forming a ring and a substituted or unsubstituted heteroaryl group having 5 to 30 carbon atoms for forming a ring, and X may be selected from a substituted or unsubstituted heteroaryl group represented by the following Formula 2:

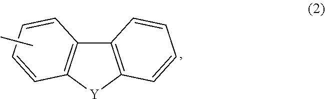

where Y may be selected from O, S and NR, and R may be selected from a substituted or unsubstituted aryl group having 6 to 30 carbon atoms for forming a ring and a substituted or unsubstituted heteroaryl group having 5 to 30 carbon atoms for forming a ring.

### CROSS-REFERENCE TO RELATED APPLICATION

This patent application claims priority to and the benefit of Japanese Patent Application No. 2014-242824, filed on Dec. 1, 2014, the entire content of which is hereby incorporated by reference.

### BACKGROUND

One or more aspects of embodiments of the present disclosure relate to a material for an organic electroluminescent device and an organic electroluminescent device using the same, and more particularly, to a material for an organic electroluminescent device having a high emission efficiency and a long life, and an organic electroluminescent device using the same.

Organic electroluminescent (EL) displays are one type of image display that is recently being actively developed. Unlike liquid crystal displays and the like, the organic EL display is a self-luminescent display in which holes and electrons injected from an anode and a cathode are recombined in an emission layer that contains luminescent material including an organic compound, and light is thus emitted from the luminescent material to achieve the display of images.

An example of an organic EL device may include an anode, a hole transport layer positioned on the anode, an emission layer positioned on the hole transport layer, an electron transport layer positioned on the emission layer, and a cathode positioned on the electron transport layer. Holes from the anode are injected via the hole transport layer into the emission layer. Meanwhile, electrons from the cathode are injected via the electron transport layer into the emission layer. The holes and the electrons injected into the emission layer are recombined to generate excitons in the emission layer. The organic EL device emits light generated by deactivated radiation during the transition of these excitons (e.g., through the radiative decay of these excitons). However, the organic EL device is not limited to the above-described configuration and may be varied in many ways.

An organic EL device utilized in a display apparatus is required to have a high efficiency and long life. For example, organic EL devices in the blue emission region may require comparatively higher driving voltages and may exhibit lower emission efficiencies than those in the green and red emission regions. The normalization, stabilization and durability of the hole transport layer have been examined in an effort to develop high efficiency, long lived organic EL devices.

Although various hole transport materials such as aromatic amine compounds have previously been used in the hole transport layer, a need to further increase the emission efficiency remains. For example, a diamine derivative has been suggested as a useful material for increasing the emission efficiency of an organic EL device in a blue emission region. The diamine derivative has also been suggested as a host material in an emission layer and as the material in a capping layer positioned on the exterior of an electrode in the organic EL. However, these diamine exhibit insufficient function as a hole transport material, and the organic EL device using the diamine derivative has an insufficient emission efficiency. Thus, an organic EL device having improved efficiency is desirable.

### SUMMARY

One or more aspects of embodiments of the present disclosure are directed toward a material for an organic EL device that has a high emission efficiency, and an organic EL device using the same.

In some embodiments, the present disclosure provides a material for an organic EL device that has a high emission efficiency in the blue emission region, and an organic EL device including the material in at least one layer positioned between an anode and a cathode of the organic EL device.

An embodiment of the present disclosure provides a material for an organic EL device represented by the following Formula 1:

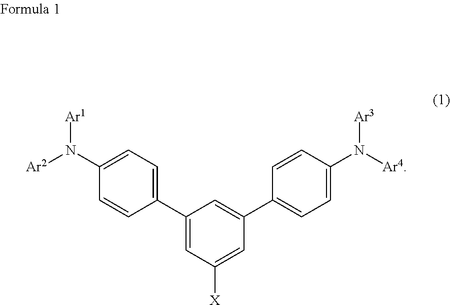

In the above Formula 1, Ar₁ to Ar₄ may each independently be selected from a substituted or unsubstituted aryl group having 6 to 30 carbon atoms for forming a ring and a substituted or unsubstituted heteroaryl group having 5 to 30 carbon atoms for forming a ring, and X may be a substituted or unsubstituted heteroaryl group represented by the following Formula 2:

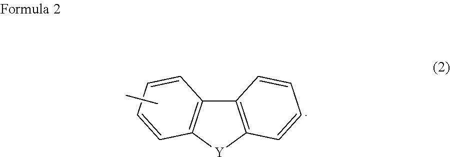

In the above Formula 2, Y may be selected from O, S and NR, and R may be selected from a substituted or unsubstituted aryl group having 6 to 30 carbon atoms for forming a ring and a substituted or unsubstituted heteroaryl group having 5 to 30 carbon atoms for forming a ring.

In one or more embodiments of the present disclosure, the material for an organic EL device includes a compound in which two amine moieties are combined (e.g., coupled) via a 1,3,5-trisubstituted benzene ring at positions 1 and 3 of the trisubstituted benzene ring. Since the two amine parts (e.g., amine moieties) are combined (e.g., coupled) via the trisubstituted benzene at these positions, conjugation in the molecule may not be wide (e.g., conjugation in the molecule may be limited), the energy gap may increase, and the emission efficiency of the organic EL device may increase. In addition, the introduction of a heteroaryl group as a substituent to the 5-position of the trisubstituted benzene may further improve the charge transport properties and emission efficiency of the organic EL device.

In one or more embodiments, Ar₁ to Ar₄ in Formula 1 may each independently be selected from a phenyl group, a biphenyl group, a naphthyl group, a terphenyl group and a phenanthryl group.

In one or more embodiments, the material for an organic EL device may enable high emission efficiency of the organic EL device.

In some embodiments, the heteroaryl group represented by Formula 2 may be a monovalent group selected from a 1-carbazolyl group, a 2-carbazolyl group, a 3-carbazolyl group, a 4-carbazolyl group, a 1-dibenzofuranyl group, a 2-dibenzofuranyl group, a 3-dibenzofuranyl group, a 4-dibenzofuranyl group, a 1-dibenzothiophenyl group, a 2-dibenzothiophenyl group, a 3-dibenzothiophenyl group and a 4-dibenzothiophenyl group.

In one or more embodiments, the material for an organic EL device may enable high emission efficiency in the organic EL device.

In one or more embodiments, the material for an organic EL device may be included in at least one layer positioned between an anode and a cathode of the organic EL device.

In one or more embodiments of the present disclosure, when the material for an organic EL device is included in at least one layer positioned between an anode and a cathode of the organic EL device, high emission efficiency may be obtained.

In one or more embodiments, the material for an organic EL device may be included in at least one layer positioned between an emission layer and an anode in the organic EL device.

In one or more embodiments, the material for an organic EL device may be included in the emission layer of the organic EL device.

In one or more embodiments, the material for an organic EL device may be included in the hole transport layer of the organic EL device.

In one or more embodiments of the present disclosure, the material for an organic EL device may be included in at least one layer positioned between an emission layer and an anode, thereby enabling high emission efficiency.

### BRIEF DESCRIPTION OF THE FIGURES

The accompanying drawings are included to provide a further understanding of the present disclosure, and are incorporated in and constitute a part of this specification. The drawings illustrate example embodiments and explain principles of the present disclosure. In the drawings:

FIG. 1 is a schematic view of an organic EL device 100 according to one or more embodiments of the present disclosure; and

FIG. 2 is a schematic view of an organic EL device 200 according to one or more embodiments of the present disclosure.

### DETAILED DESCRIPTION

According to one or more embodiments of the present disclosure, a compound having two amine moieties (hereinafter referred to as a “diamine compound”) combined (e.g., coupled) to a 1,3,5-trisubstituted benzene at positions 1 and 3 of the trisubstituted benzene ring, and further including a heteroaryl group coupled to the trisubstituted benzene at position 5 of the trisubstituted benzene ring, may increase the charge transport properties of the layer using the diamine compound and thus may enable high emission efficiency of the organic EL device.

Hereinafter, a material for an organic EL device and an organic EL device including the same, according to the one or more embodiments of the present disclosure, will be described in more detail with reference to the accompanying drawings. The material for an organic EL device and the organic EL device including the same as described in embodiments of the present disclosure may, however, be embodied in different forms and should not be construed as being limited to the embodiments set forth herein. In the description and drawings, elements having substantially the same function are designated by the same reference numerals, and repeated explanation thereof will not be provided.

According to one or more embodiments of the present disclosure, a material for an organic EL device may be a diamine compound represented by the following Formula 1:

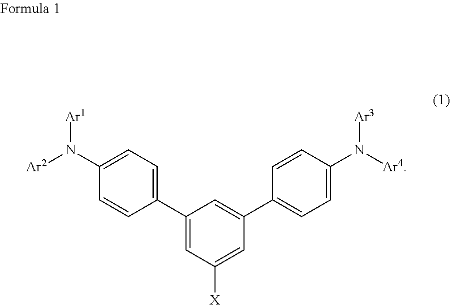

In the above Formula 1, Ar₁ to Ar₄ may be each independently selected from a substituted or unsubstituted aryl group having 6 to 30 carbon atoms for forming a ring and a substituted or unsubstituted heteroaryl group having 5 to 30 carbon atoms for forming a ring, and X may be a substituted or unsubstituted heteroaryl group represented by the following Formula 2:

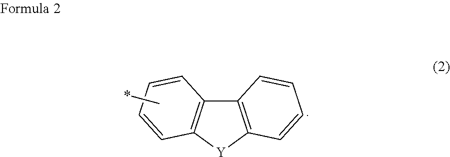

In the above Formula 2, Y may be selected from O, S and NR, and R may be selected from a substituted or unsubstituted aryl group having 6 to 30 carbon atoms for forming a ring and a substituted or unsubstituted heteroaryl group having 5 to 30 carbon atoms for forming a ring. As used herein, the statement “atoms for forming a ring” may refer to “ring-forming atoms”. In addition, * represents a binding site of Formula 2 to the trisubstituted benzene in Formula 1.

In the above Formula 1, non-limiting examples of the aryl group having 6 to 30 carbon atoms for forming a ring used, for example, for Ar₁ to Ar₄ may include a phenyl group, a naphthyl group, an anthracenyl group, a phenanthryl group, a biphenyl group, a terphenyl group, a quaterphenyl group, a fluorenyl group, a triphenylene group, a biphenylene group, a pyrenyl group, a benzofluoranthenyl group, a glyceryl group, a phenylnaphthyl group, a naphthyl phenyl group, and the like.

Non-limiting examples of the heteroaryl group having 5 to 30 carbon atoms for forming a ring used, for example, for Ar₁ to Ar₄ may include a pyridyl group, a quinolinyl group, a quinoxalinyl group, a phenanthrolinyl group, a pyrrolyl group, an indolyl group, a carbazolyl group, a benzoimidazolyl group, an oxazolyl group, an oxadiazolyl group, a triazolyl group, a furanyl group, a benzofuranyl group, a dibenzofuranyl group, a thiophenyl group, a benzothiophenyl group, a dibenzothiophenyl group, a silole group, a benzosilole group, a dibenzosilole group, and the like.

Non-limiting examples of the substituents of, for example, Ar₁ to Ar₄ may include an alkyl group having 1 to 6 carbon atoms, an alkoxy group having 1 to 6 carbon atoms, a phenyl group, and the like. The alkyl group having 1 to 6 carbon atoms may include, for example, a methyl group, an ethyl group, a n-propyl group, an i-propyl group, a n-butyl group, a s-butyl group, a t-butyl group, a n-pentyl group, a n-hexyl group, a c-propyl group, a c-butyl group, a c-pentyl group, a c-hexyl group, and the like. Non-limiting examples of the alkoxy group having 1 to 6 carbon atoms may include, for example, a methoxy group, an ethoxy group, a n-propoxy group, an i-propoxy group, a n-butoxy group, a s-butoxy group, a t-butoxy group, a n-pentoxy group, a n-hexoxy group, a c-propoxy group, a c-butoxy group, a c-pentoxy group, a c-hexoxy group, and the like.

As described above in Formula 1, X may be a heteroaryl group represented by Formula 2, and in Formula 2, Y may be selected from O, S and NR.

Non-limiting examples of the aryl group having 6 to 30 carbon atoms for forming a ring and a heteroaryl group having 5 to 30 carbon atoms for forming a ring used, for example, for R may be the same as those provided in connection with the aryl group having 6 to 30 carbon atoms for forming a ring and the heteroaryl group having 5 to 30 carbon atoms for forming a ring used for Ar₁ to Ar₄.

The heteroaryl group represented by Formula 2, that is, X in Formula 1, may be a monovalent group (e.g., monovalent species) selected from, for example, a 1-carbazolyl group, a 2-carbazolyl group, a 3-carbazolyl group, a 4-carbazolyl group, a 1-dibenzofuranyl group, a 2-dibenzofuranyl group, a 3-dibenzofuranyl group, a 4-dibenzofuranyl group, a 1-dibenzothiophenyl group, a 2-dibenzothiophenyl group, a 3-dibenzothiophenyl group and a 4-dibenzothiophenyl group.

In one or more embodiments, one or more substituents of the heteroaryl group represented by Formula 2 may be selected from the same example substituents as those used in connection with Ar₁ to Ar₄.

In one or more embodiments of the present disclosure, in the material for an organic EL device represented by Formula 1, carbon atom (C) of the trisubstituted benzene may be combined (e.g., coupled) to a ring-forming carbon atom (C) of the heteroaryl group represented by Formula 2 (where X in Formula 1 represents the heteroaryl group of Formula 2) and not to Y (that is a heteroatom).

In one of more embodiments of the present disclosure, the material for an organic EL device includes a diamine compound in which two amine moieties are combined (e.g., coupled) via a 1,3,5-trisubstituted benzene at positions 1 and 3 of the trisubstituted benzene ring. Since the two amine parts (e.g., amine moieties) are combined (e.g., coupled) via the trisubstituted benzene at these positions, conjugation in the molecule may not be wide (e.g., conjugation in the molecule may be limited), the energy gap may increase, and the emission efficiency of the organic EL device may be improved. The inclusion of a heteroaryl group at the 5-position of the trisubstituted benzene may further improve the charge transport properties and the emission efficiency of the organic EL device.

In one or more embodiments, the material for an organic EL device may be represented by at least one selected from the following Compounds 1 to 7:

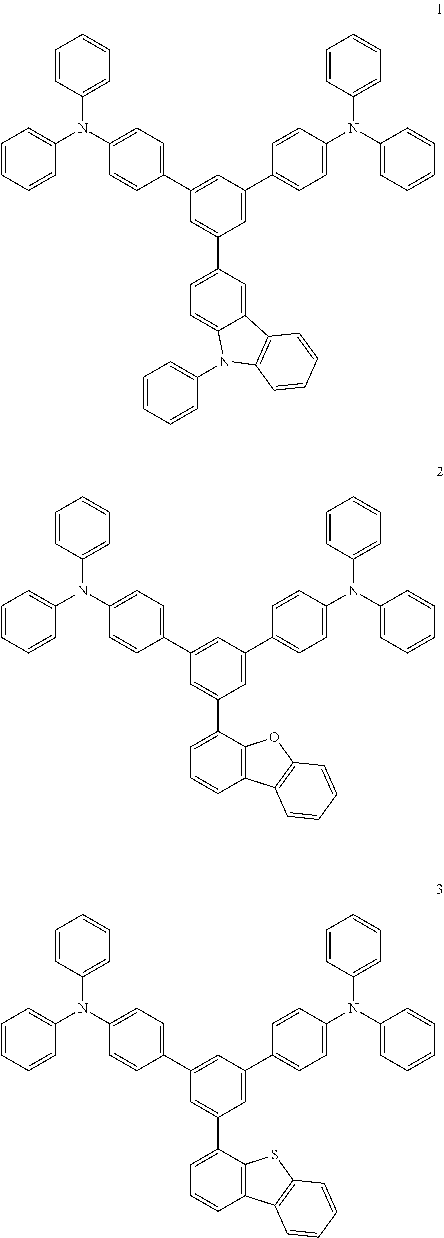 [SMILES: c1ccc(N(c2ccccc2)c2ccc(-c3cc(-c4ccc(N(c5ccccc5)c5ccccc5)cc4)cc(-c4ccc5c(c4)c4ccccc4n5-c4ccccc4)c3)cc2)cc1 ; c1ccc(N(c2ccccc2)c2ccc(-c3cc(-c4ccc(N(c5ccccc5)c5ccccc5)cc4)cc(-c4cccc5c4oc4ccccc45)c3)cc2)cc1 ; c1ccc(N(c2ccccc2)c2ccc(-c3cc(-c4ccc(N(c5ccccc5)c5ccccc5)cc4)cc(-c4cccc5c4sc4ccccc45)c3)cc2)cc1]

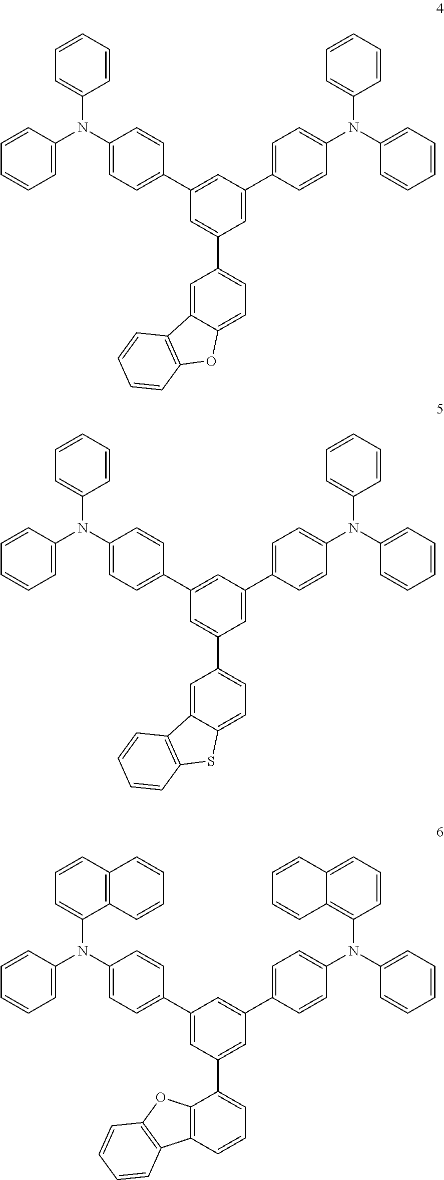 [SMILES: c1ccc(N(c2ccccc2)c2ccc(-c3cc(-c4ccc(N(c5ccccc5)c5ccccc5)cc4)cc(-c4ccc5oc6ccccc6c5c4)c3)cc2)cc1 ; c1ccc(N(c2ccccc2)c2ccc(-c3cc(-c4ccc(N(c5ccccc5)c5ccccc5)cc4)cc(-c4ccc5sc6ccccc6c5c4)c3)cc2)cc1 ; c1ccc(N(c2ccc(-c3cc(-c4ccc(N(c5ccccc5)c5cccc6ccccc56)cc4)cc(-c4cccc5c4oc4ccccc45)c3)cc2)c2cccc3ccccc23)cc1]

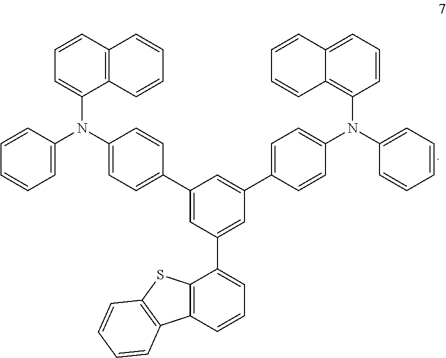 [SMILES: c1ccc(N(c2ccc(-c3cc(-c4ccc(N(c5ccccc5)c5cccc6ccccc56)cc4)cc(-c4cccc5c4sc4ccccc45)c3)cc2)c2cccc3ccccc23)cc1]

In one or more embodiments of the present disclosure, the material for an organic EL device may be included in at least one layer selected from a plurality of organic layers forming the organic EL device. For example, the material may be included in at least one layer positioned between an emission layer and an anode in the organic EL device.

As described above, in one or more embodiments of the present disclosure, the material for an organic EL device relates to a diamine compound in which two amine moieties are combined (e.g., coupled) via a 1,3,5-trisubstituted benzene moiety at positions 1 and 3 of the trisubstituted benzene ring. Since the two amine parts (e.g., amine moieties) are combined (e.g., coupled) via the trisubstituted benzene at those positions, conjugation in the molecule may not be wide (e.g., conjugation in the molecule may be limited), the energy gap may increase, and the emission efficiency of the organic EL device may be improved. The inclusion of a heteroaryl group at the 5-position of the trisubstituted benzene may further improve the charge transport properties and the emission efficiency of the organic EL device.

In one or more embodiments, the material for an organic EL device is not limited to inclusion in a layer positioned between an emission layer and an anode of the organic EL device, and may be used as a material in an emission layer.

### (Organic EL Device)

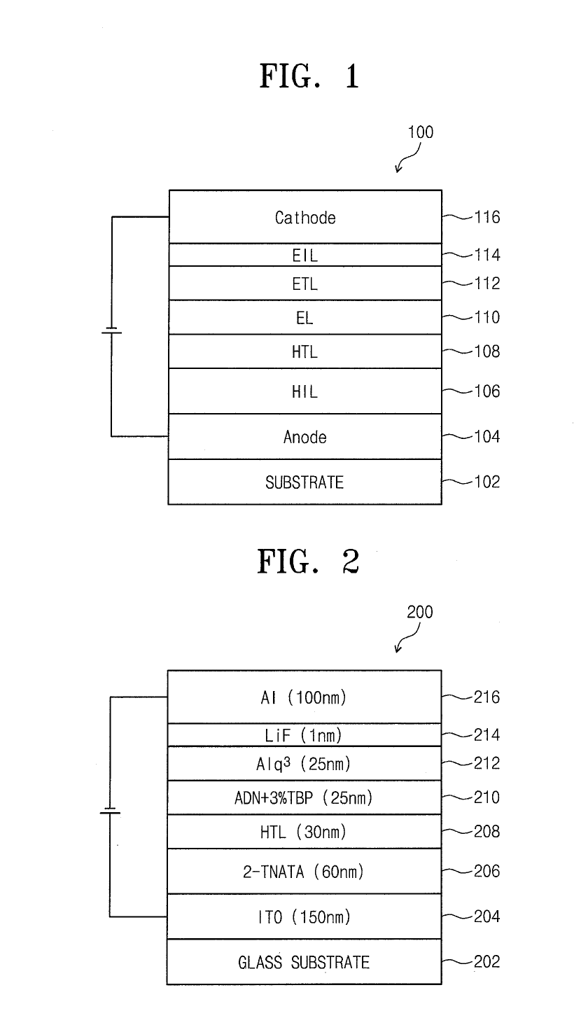

An organic EL device using the material for an organic EL device according to one or more embodiments of the present disclosure will now be described with reference to the figures. FIG. 1 is a schematic view illustrating an organic EL device 100 according to an embodiment of the present disclosure. The organic EL device 100 may include, for example, a substrate 102, an anode 104, a hole injection layer 106, a hole transport layer 108, an emission layer 110, an electron transport layer 112, an electron injection layer 114 and a cathode 116. In one or more embodiments of the present disclosure, the material for an organic EL device may be included in at least one layer selected from a plurality of layers (herein also referred to as “stacking layers”) positioned between the emission layer and the anode of the organic EL device.

An embodiment using the material for an organic EL device in the hole transport layer 108 will now be described.

The substrate 102 may be a transparent glass substrate, a semiconductor substrate formed using silicon, a flexible substrate of a resin, and/or the like.

The anode 104 may be positioned on the substrate 102 and may be formed using, for example, indium tin oxide (ITO), indium zinc oxide (IZO), and/or the like.

The hole injection layer (HIL) 106 may be positioned on the anode 104, may include any suitable material, and may be formed to a thickness within a range of about 10 nm to about 150 nm. Non-limiting examples of the hole injection material may include triphenylamine-containing polyether ketone (TPAPEK), 4-isopropyl-4′-methyldiphenyliodonium tetrakis(pentafluorophenyl)borate (PPBI), N,N′-diphenyl-N,N′-bis-[4-(phenyl-m-tolyl-amino)-phenyl]-phenyl-4,4′-diamine (DNTPD), a phthalocyanine compound such as copper phthalocyanine, 4,4′,4″-tris(3-methylphenylphenylamino)triphenylamine (m-MTDATA), 4,4′,4″-tris{N,N-diphenylamino}triphenylamine (TDATA), 4,4′,4″-tris(N,N-2-naphthylphenylamino)triphenylamine (2-TNATA), polyaniline/dodecylbenzenesulfonic acid (PANI/DBSA), poly(3,4-ethylenedioxythiophene)/poly(4-styrenesulfonate) (PEDOT/PSS), polyaniline/camphorsulfonic acid (PANI/CSA), polyaniline/poly(4-styrenesulfonate) (PANI/PSS), and the like.

The hole transport layer (HTL) 108 may be formed on the hole injection layer 106 to a thickness within a range of about 10 nm to about 150 nm using the material for an organic EL device according to one or more embodiments of the present disclosure.

In some embodiments, the material for an organic EL device may be used as a host material of the emission layer (EL) 110, and in this case, the hole transport layer 108 may be formed using any suitable hole transport material. Non-limiting examples of the hole transport material may include a carbazole derivative (such as 1,1-bis[(di-4-tolylamino)phenyl]cyclohexane (TAPC), N-phenylcarbazole, polyvinyl carbazole, and/or the like), N,N′-bis(3-methylphenyl)-N,N′-diphenyl-[1,1-biphenyl]-4,4′-diamine (TPD), 4,4′,4″-tris(N-carbazolyl)triphenylamine (TCTA), N,N′-di(1-naphthyl)-N,N′-diphenylbenzidine (NPB), and the like. In some embodiments, the hole transport layer 108 may be formed by combining any suitable hole transport material with the material for an organic EL device according to one or more embodiments of the present disclosure.

The emission layer (EL) 110 may be formed on the hole transport layer 108 using any suitable host material to a thickness within a range of about 10 nm to about 60 nm. Non-limiting examples of the host material used in the emission layer 110 may include tris(8-quinolinolato)aluminum (Alq3), 4,4′-N,N′-dicarbazole-biphenyl (CBP), poly(n-vinylcarbazole) (PVK), 9,10-di(naphthalene-2-yl)anthracene (ADN), 4,4′,4″-tris(N-carbazolyl)triphenylamine (TCTA), 1,3,5-tris(N-phenylbenzimidazole-2-yl)benzene (TPBI), 3-tert-butyl-9,10-di(naphtho-2-yl)anthracene (TBADN), distyrylarylene (DSA), 4,4′-bis(9-carbazole)-2,2′-dimethyl-biphenyl (dmCBP), and the like.

The emission layer 110 may further include a dopant material, and non-limiting examples of the dopant material used in the emission layer 110 may include a styryl derivative (e.g., 1,4-bis[2-(3-N-ethylcarbazolyl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalene-2-yl)vinyl)phenyl-N-phenylbenzenamine (N-BDAVBi)), perylene and/or derivatives thereof (e.g., 2,5,8,11-tetra-t-butylperylene (TBPe)), pyrene and/or derivatives thereof (e.g., 1,1-dipyrene, 1,4-dipyrenylbenzene, 1,4-bis(N,N-diphenylamino)pyrene), and the like.

The electron transport layer (ETL) 112 may be formed on the emission layer 110 to a thickness within a range of about 15 nm to about 50 nm. Non-limiting examples of a material used for the electron transport layer 112 include Tris(8-hydroxyquinolinato)aluminum(Alq3) and a material having a nitrogen-containing aromatic ring (e.g., a material including a pyridine ring such as 1,3,5-tri[(3-pyridyl)-phen-3-yl]benzene, a material including a triazine ring such as 2,4,6-tris(3′-(pyridine-3-yl)biphenyl-3-yl)1,3,5-triazine, and/or a material including an imidazole derivative such as 2-(4-N-phenylbenzoimidazolyl-1-ylphenyl)-9,10-dinaphthylanthracene).

The electron injection layer (EIL) 114 may be formed on the electron transport layer 112 to a thickness within a range of about 0.3 nm to about 9 nm using, for example, a material including lithium fluoride (LiF), lithium-8-quinolinolato (Liq), and/or the like.

The cathode 116 may be positioned on the electron injection layer 114 and may be formed using, for example, metals such as aluminum (Al), silver (Ag), lithium (Li), magnesium (Mg), calcium (Ca), and/or the like, mixtures thereof, and/or transparent materials such as ITO, IZO, and/or the like.

In one or more embodiments, each electrode and each layer forming the organic EL device according to embodiments of the present disclosure may be formed by one or more suitable layer forming methods such as vacuum evaporation, sputtering, and/or other various coating methods, depending on the material for forming each electrode or layer.

In the organic EL device 100 according to embodiments of the present disclosure, a hole transport layer realizing the high efficiency of the organic EL device may be formed by using the material for an organic EL device according to embodiments of the present disclosure.

In the organic EL device 100 according to embodiments of the present disclosure, the material for an organic EL device may be used in a hole injection layer or as a host material in an emission layer. As described above, high organic EL device efficiencies may be realized by including the material for an organic EL device in at least one layer of a plurality of organic layers forming the organic EL device.

In addition, in one or more embodiments, the material for an organic EL device may be applied in an active matrix type organic EL device (e.g., in an active matrix organic EL device) using a thin film transistor (TFT).

Hereinafter, one or more embodiments of the methods of synthesizing the material for an organic EL device and the manufacturing of the organic EL device will be explained in more detail. However, the following examples are provided for illustrative purposes, and the scope of the present disclosure is not limited thereto.

### (Preparation Method)

In one or more embodiments, the material for an organic EL device may be synthesized, for example, as follows:

### Synthetic Method of Compound 2

### (Synthesis of Compound A)

Compound A, illustrated below, was synthesized according to the following scheme. Under an argon atmosphere, 11.14 g of 1,3,5-tribromobenzene, 5.00 g of dibenzofuran-4-boronic acid, 1.36 g of tetrakis(triphenylphosphine)palladium(0), and 5.00 g of sodium carbonate were added to a 1 L, three-necked flask, followed by stirring in 250 mL of a solvent mixture of toluene/water/ethanol (10:1:1) at about 80° C. for about 5 hours. Water was added to the reaction, the organic layer was separated therefrom, and the solvents were distilled. The crude product thus obtained was separated by silica gel column chromatography (using toluene/hexane) to produce 4.74 g of Compound A as a white solid (Yield 50%). The molecular weight of the product was measured by Fast Atom Bombardment Mass Spectrometry (FAB-MS) to be 400, the chemical formula thereof was taken to be C₁₈H₁₀Br₂O, and the product was confirmed as Compound A.

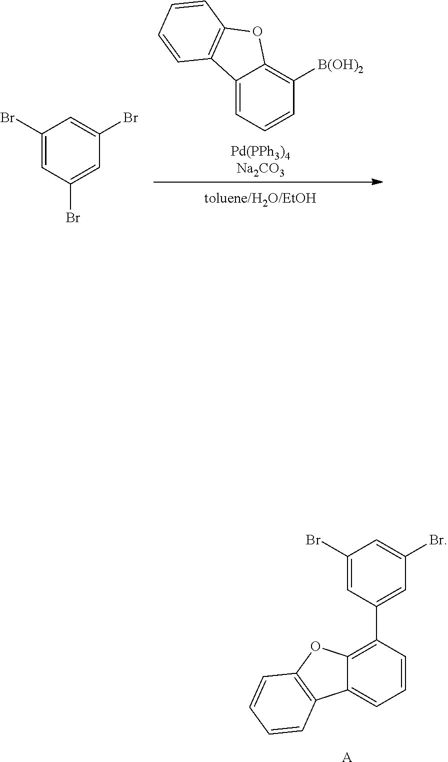

### (Synthesis of Compound 2)

Compound 2 was synthesized according to the following scheme. Under an argon atmosphere, 4.74 g of Compound A, 7.50 g of 4-(diphenylamino)phenylboronic acid, 1.36 g of tetrakis(triphenylphosphine)palladium(0), and 5.00 g of sodium carbonate were added to a 500 mL, three-necked flask, followed by stirring in 120 mL of a solvent mixture of toluene/water/ethanol (10:1:1) at about 80° C. for about 6 hours. Water was added to the reaction, the organic layer was separated therefrom, and the solvents were distilled. The crude product thus obtained was separated by silica gel column chromatography (using toluene/hexane) to produce 6.12 g of Compound 2 as a white solid (Yield 71%). The molecular weight of the product was measured by FAB-MS to be 730, the chemical formula thereof was taken to be C₅₄H₃₈N₂O, and the product was confirmed as Compound 2.

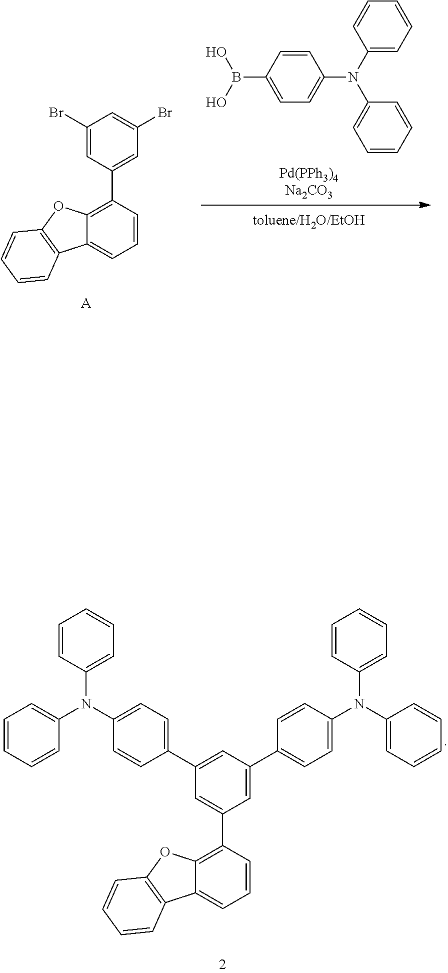

In one or more embodiments, additional example material for an organic EL device may be synthesized as follows:

### Synthesis Method of Compound 4

### (Synthesis of Compound B)

Compound B, illustrated below, was synthesized according to the following scheme. Under an argon atmosphere, 10.00 g of 2-bromodibenzofuran, 12.33 g of bis(pinacolato)diborane, 1.65 g of [1,1′-bis(diphenylphosphino)ferrocene] palladium(II)dichloride.dichloromethane adduct, and 11.95 g of potassium acetate were added to a 500 mL, three-necked flask, followed by stirring in 200 mL of dehydrated 1,4-dioxane at about 100° C. for about 2 hours. Water was added to the reaction, extraction with ethyl acetate was performed, the organic layers were collected, and the solvents were distilled. The crude product thus obtained was separated by silica gel column chromatography (using toluene/hexane) to produce 10.64 g of Compound B as a white solid (Yield 89%). The molecular weight of the product was measured by FAB-MS to be 294, the chemical formula thereof was taken to be C₁₈H₁₉BO₃, and the product was confirmed as Compound B.

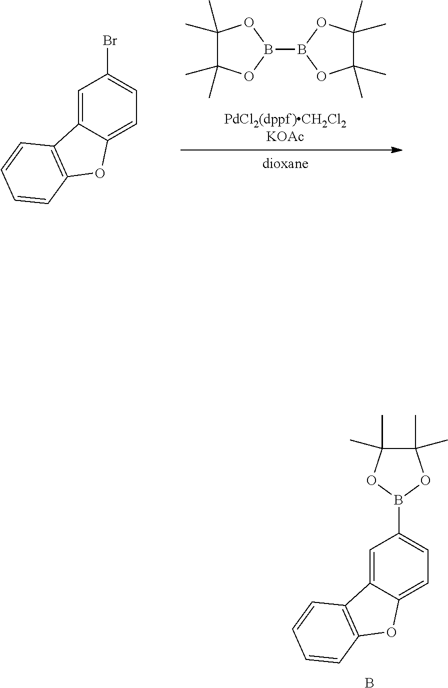

### (Synthesis of Compound C)

Compound C, illustrated below, was synthesized according to the following scheme. Under an argon atmosphere, 17.08 g of 1,3,5-tribromobenzene, 10.64 g of Compound B, 2.09 g of tetrakis(triphenylphosphine)palladium(0), and 15.36 g of potassium phosphate were added to a 1 L, three-necked flask, followed by stirring in 400 mL of a solvent mixture of toluene/water/ethanol (10:1:1) at about 80° C. for about 6 hours. Water was added to the reaction, the organic layer was separated therefrom, and the solvents were distilled. The crude product thus obtained was separated by silica gel column chromatography (using toluene/hexane) to produce 7.56 g of Compound C as a white solid (Yield 52%). The molecular weight of the product was measured by FAB-MS to be 400, the chemical formula thereof was taken to be C₁₈H₁₀Br₂O, and the product was confirmed as Compound C.

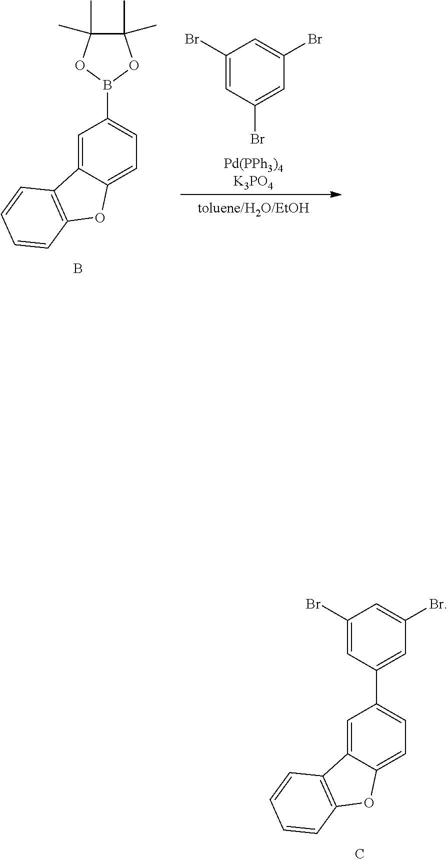

### (Synthesis of Compound 4)

Compound 4 was synthesized according to the following scheme. Under an argon atmosphere, 3.80 g of Compound C, 6.01 g of 4-(diphenylamino)phenylboronic acid, 1.09 g of tetrakis(triphenylphosphine)palladium(0), and 4.01 g of sodium carbonate were added to a 500 mL, three-necked flask, followed by stirring in 120 mL of a solvent mixture of toluene/water/ethanol (10:1:1) at about 80° C. for about 3 hours. Water was added to the reaction, the organic layer was separated therefrom, and the solvents were distilled. The crude product thus obtained was separated by silica gel column chromatography (toluene/hexane) to produce 6.03 g of Compound 4 as a white solid (Yield 87%). The molecular weight of the product was measured by FAB-MS to be 730, the chemical formula thereof was taken to be C₅₄H₃₈N₂O, and the product was confirmed as Compound 4.

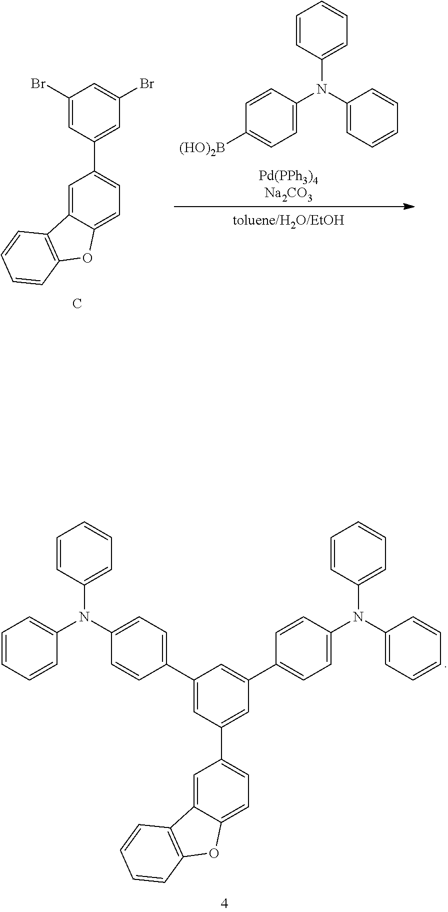

In one or more embodiments, additional example material for an organic EL device may be synthesized as follows:

### Synthetic Method of Compound 5

### (Synthesis of Compound D)

Compound D, illustrated below, was synthesized according to the following synthetic scheme. Under an argon atmosphere, 21.28 g of 1,3,5-tribromobenzene, 10.33 g of dibenzofuran-4-boronic acid, 2.03 g of tetrakis(triphenylphosphine)palladium(0) and 7.44 g of sodium carbonate were added to a 1 L, three-necked flask, followed by stirring in 400 mL of a solvent mixture of toluene/water/ethanol (10:1:1) at about 80° C. for about 4 hours. Water was added to the reaction, the organic layer was separated therefrom, and the solvents were distilled. The crude product thus obtained was recrystallized from toluene to produce 3.64 g of Compound D as a white solid (Yield 25%). The molecular weight of the product was measured by FAB-MS to be 416, the chemical formula thereof was taken to be C₁₈H₁₀Br₂S, and the product was confirmed as Compound D.

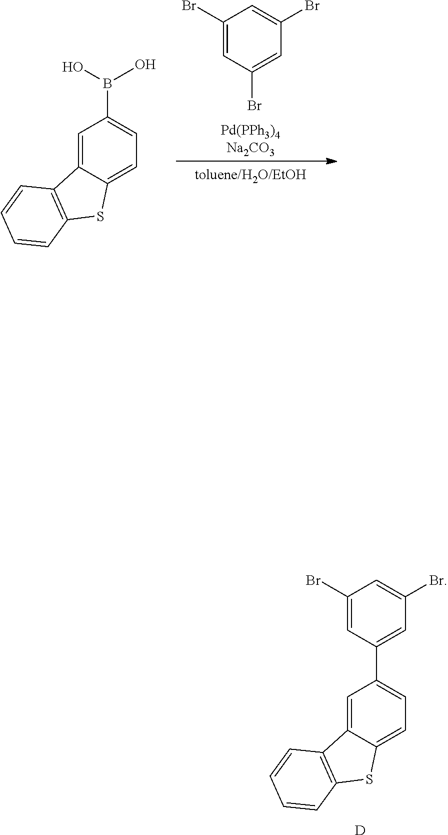

### (Synthesis of Compound 5)

Compound 5 was synthesized according to the following synthetic scheme. Under an argon atmosphere, 3.60 g of Compound D, 5.48 g of 4-(diphenylamino)phenylboronic acid, 0.99 g of tetrakis(triphenylphosphine)palladium(0), and 3.65 g of sodium carbonate were added to a 500 mL, three-necked flask, followed by stirring in 110 mL of a solvent mixture of toluene/water/ethanol (10:1:1) at about 80° C. for about 3 hours. Water was added to the reaction, the organic layer was separated therefrom, and the solvents were distilled. The crude product thus obtained was separated by silica gel column chromatography (toluene) and recrystallized from toluene to produce 4.77 g of Compound 5 as a white solid (Yield 74%). The molecular weight of the product was measured by FAB-MS to be 746, the chemical formula thereof was taken to be C₅₄H₃₈N₂S, and the target product was confirmed as Compound 5.

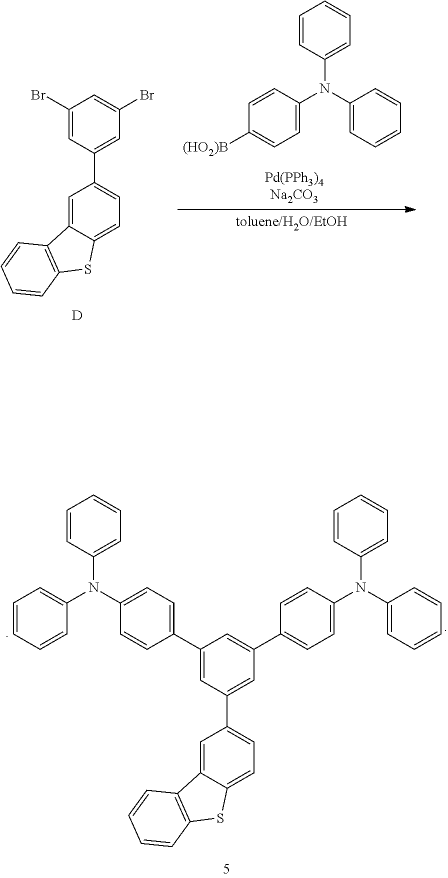

In one or more embodiments, additional example material for an organic EL device may be synthesized as follows.

### Synthetic Method of Compound 3

### (Synthesis of Compound E)

Compound E, illustrated below, was synthesized according to the following scheme. 20.0 g of 1,3,5-tribromobenzene, 36.7 g of 4-(diphenylamino)phenylboronic acid, 54 mL of toluene, 27 mL of ethanol and 64 mL of a 2M sodium carbonate aqueous solution were added to a reaction vessel, and the inner gas of the reaction vessel was substituted with argon. Under an argon atmosphere, 2.2 g of Pd(PPh₃)₄ was added thereto, followed by heating and refluxing the mixture for about 1 hour while stirring. After air cooling the resultant, the organic layer was extracted therefrom, the resultant was dried with magnesium sulfate, and filtered. The filtrate was concentrated using a rotary evaporator under reduced pressure. The crude product thus obtained was separated by silica gel column chromatography (development solvent: dichloromethane/hexane), and the solid thus obtained was recrystallized from toluene/hexane to produce 16.4 g of Compound E as a powdery white solid (Yield 40%). The molecular weight of the product was measured by FAB-MS to be 642, the chemical formula thereof was taken to be C₄₂H₃₁BrN₂, and the target product was confirmed as Compound E.

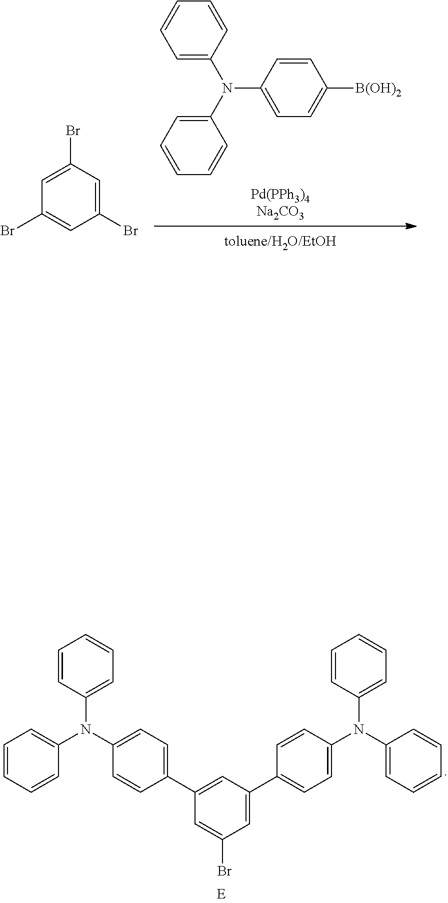 [SMILES: Brc1cc(Br)cc(Br)c1 ; OB(O)c1ccc(N(c2ccccc2)c2ccccc2)cc1 ; Brc1cc(-c2ccc(N(c3ccccc3)c3ccccc3)cc2)cc(-c2ccc(N(c3ccccc3)c3ccccc3)cc2)c1]

### (Synthesis of Compound 3)

Compound 3 was synthesized according to the following scheme. 7.0 g of Compound E, 2.7 g of dibenzothiophene-4-boronic acid, 44 mL of toluene, 22 mL of ethanol and 11 mL of a 2 M sodium carbonate aqueous solution were added to a reaction vessel, and the inner gas of the reaction vessel was substituted with argon. Under an argon atmosphere, 0.4 g of Pd(PPh₃)₄ was added thereto, followed by heating and refluxing the mixture for about 2 hours while stirring. After air cooling the resultant, the organic layer was extracted therefrom, the resultant was dried with magnesium sulfate, and filtered. The filtrate was concentrated using a rotary evaporator under reduced pressure. The crude product thus obtained was separated by silica gel column chromatography (development solvent: toluene/hexane), and the solid thus obtained was recrystallized from toluene/hexane to produce 5.7 g of Compound 3 as a pale yellow crystalline solid (Yield 70%). The molecular weight of the product was measured by FAB-MS to be 746, the chemical formula thereof was taken to be C₅₄H₃₈N₂S, and the target product was confirmed as Compound 3.

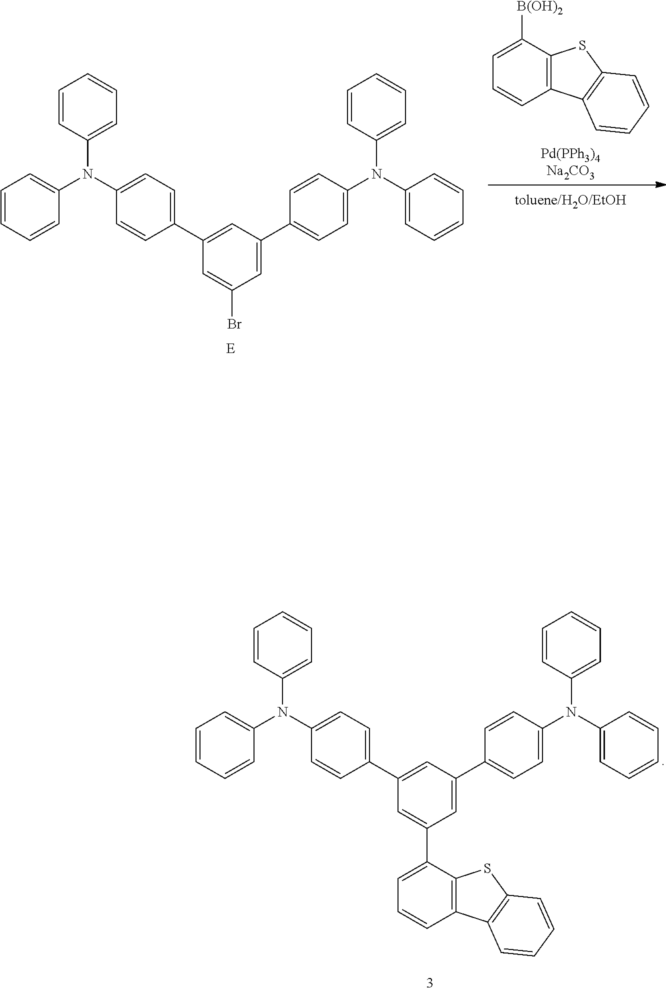 [SMILES: Brc1cc(-c2ccc(N(c3ccccc3)c3ccccc3)cc2)cc(-c2ccc(N(c3ccccc3)c3ccccc3)cc2)c1 ; OB(O)c1cccc2c1sc1ccccc12 ; c1ccc(N(c2ccccc2)c2ccc(-c3cc(-c4ccc(N(c5ccccc5)c5ccccc5)cc4)cc(-c4cccc5c4sc4ccccc45)c3)cc2)cc1]

In one or more embodiments, additional example material for an organic EL device may be synthesized as follows:

### Synthetic Method of Compound 6

### (Synthesis of Compound G)

Compound G, illustrated below, was synthesized according to the following scheme. 15.4 g of boronic ester (Compound F), 4.4 g of 1,3-dibromo-5-chlorobenzene, 176 mL of toluene, 73 mL of ethanol and 37 mL of a 2M sodium carbonate aqueous solution were added to a reaction vessel, and the inner gas of the reaction vessel was substituted with argon. Under an argon atmosphere, 2.5 g of Pd(PPh₃)₄ was added thereto, followed by heating and stirring the mixture at about 85° C. for about 5 hours. After air cooling the resultant, the organic layer was extracted therefrom, the resultant was dried with magnesium sulfate, and filtered. The filtrate was concentrated using a rotary evaporator under reduced pressure. The crude product thus obtained was separated by silica gel column chromatography (development solvent: dichloromethane/hexane), and the solid thus obtained was recrystallized from dichloromethane/ethanol to produce 10.9 g of Compound Gas a powdery pale yellow solid (Yield 95%). The molecular weight of the product was measured by FAB-MS to be 698, the chemical formula thereof was taken to be C₅₀H₃₅ClN₂, and the target product was confirmed as Compound G.

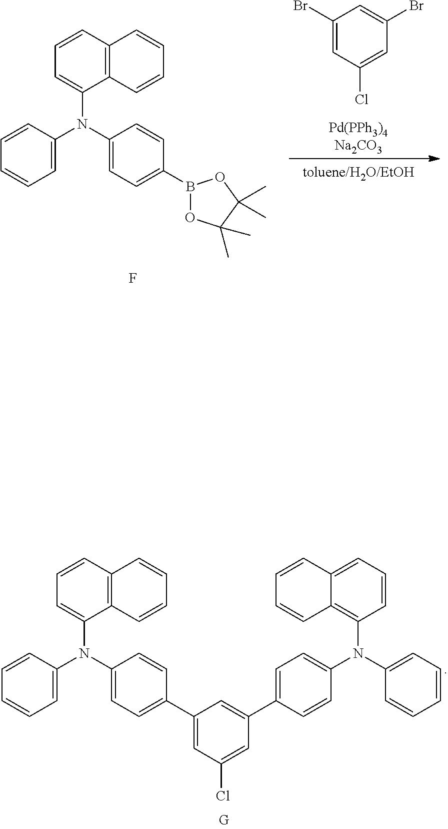

### (Synthesis of Compound 6)

Compound 6 was synthesized according to the following scheme. 4.80 g of Compound G, 2.18 g of dibenzofuran-4-boronic acid, 2.91 g of potassium phosphate, 27.5 mL of toluene, and 2.8 mL of water were added to a reaction vessel, and the inner gas of the reaction vessel was substituted with argon. Under an argon atmosphere, 0.05 g of palladium(II) acetate and 0.17 g of 2-dicyclohexylphosphino-2′,6′-dimethoxybiphenyl (SPhos) were added thereto, followed by heating and stirring the mixture at about 100° C. for about 3 hours. After air cooling the resultant, the organic layer was extracted therefrom, the resultant was dried with magnesium sulfate, and filtered. The filtrate was concentrated using a rotary evaporator under reduced pressure. The crude product thus obtained was separated by silica gel column chromatography (development solvent: dichloromethane/hexane), and the solid thus obtained was recrystallized from dichloromethane/ethanol to produce 5.56 g of Compound 6 as a white solid (Yield 97%). The molecular weight of the product was measured by FAB-MS to be 830, the chemical formula thereof was taken to be C₆₂H₄₂N₂O, and the target product was confirmed as Compound 6.

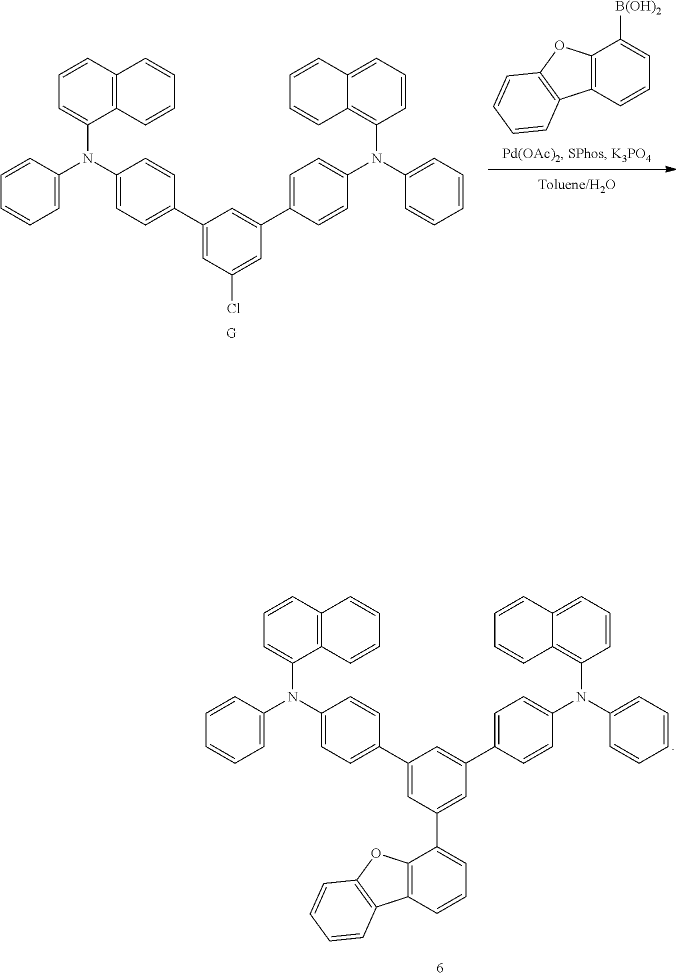 [SMILES: Clc1cc(-c2ccc(N(c3ccccc3)c3cccc4ccccc34)cc2)cc(-c2ccc(N(c3ccccc3)c3cccc4ccccc34)cc2)c1 ; OB(O)c1cccc2c1oc1ccccc12 ; c1ccc(N(c2ccc(-c3cc(-c4ccc(N(c5ccccc5)c5cccc6ccccc56)cc4)cc(-c4cccc5c4oc4ccccc45)c3)cc2)c2cccc3ccccc23)cc1]

In one or more embodiments, additional example material for an organic EL device may be synthesized as follows:

### Synthetic Method of Compound 7

### (Synthesis of Compound 7)

Compound 7 was synthesized from Compound G, above, according to the following scheme. 4.80 g of Compound G, 2.35 g of dibenzofuran-4-boronic acid, 2.91 g of potassium phosphate, 27.5 mL of toluene, and 2.8 mL of water were added to a reaction vessel, and the inner gas of the reaction vessel was substituted with argon. Under an argon atmosphere, 0.05 g of palladium(II) acetate and 0.17 g of SPhos were added thereto, followed by heating and stirring the mixture at about 100° C. for about 3 hours. After air cooling the resultant, the organic layer was extracted therefrom, the resultant was dried with magnesium sulfate, and filtered. The filtrate was concentrated using a rotary evaporator under reduced pressure. The crude product thus obtained was separated by silica gel column chromatography (development solvent: dichloromethane/hexane), and the solid thus obtained was recrystallized from dichloromethane/ethanol to produce 5.40 g of Compound 7 as a white solid (Yield 93%). The molecular weight of the product was measured by FAB-MS to be 846, the chemical formula thereof was taken to be C₆₂H₄₂N₂S, and the target product was confirmed as Compound 7.

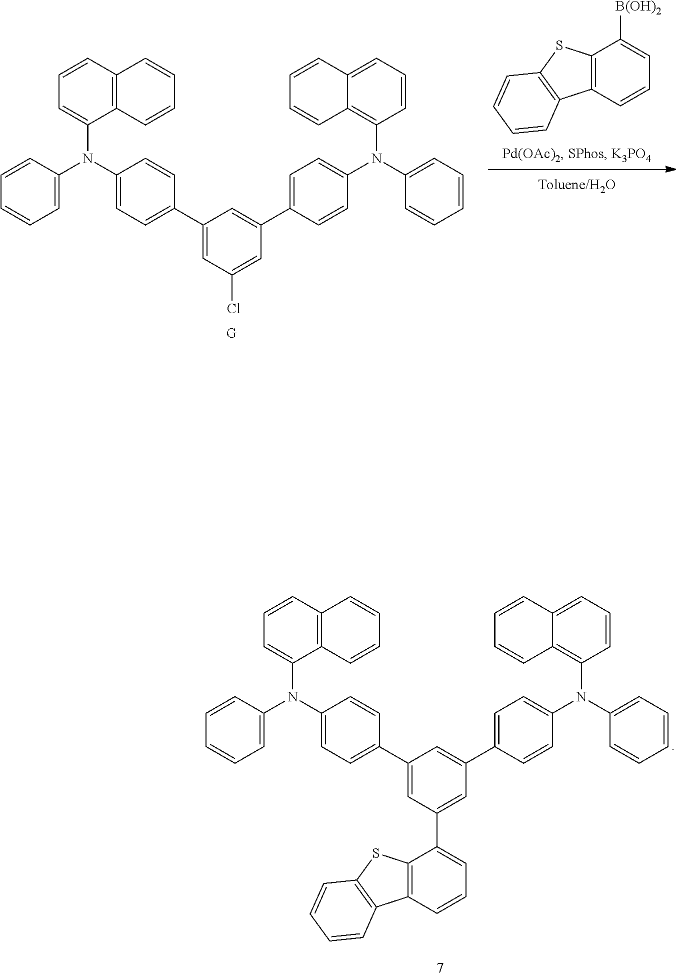 [SMILES: Clc1cc(-c2ccc(N(c3ccccc3)c3cccc4ccccc34)cc2)cc(-c2ccc(N(c3ccccc3)c3cccc4ccccc34)cc2)c1 ; OB(O)c1cccc2c1sc1ccccc12 ; c1ccc(N(c2ccc(-c3cc(-c4ccc(N(c5ccccc5)c5cccc6ccccc56)cc4)cc(-c4cccc5c4sc4ccccc45)c3)cc2)c2cccc3ccccc23)cc1]

In one or more embodiments, additional example material for an organic EL device may be synthesized as follows.

### Synthetic Method of Compound 1

### (Synthesis of Compound 1)

Compound 1 was synthesized from Compound E, above, according to the following scheme. 7.0 g of Compound E, 3.4 g of N-phenylcarbazole-3-boronic acid, 44 mL of toluene, 22 mL of ethanol and 11 mL of a 2M sodium carbonate aqueous solution were added to a reaction vessel, and the inner gas of the reaction vessel was substituted with argon. Under an argon atmosphere, 0.4 g of Pd(PPh₃)₄ was added thereto, followed by heating and refluxing the mixture for about 2 hours while stirring. After air cooling the resultant, the organic layer was extracted therefrom, the resultant dried with magnesium sulfate, and filtered. The filtrate was concentrated using a rotary evaporator under reduced pressure. The crude product thus obtained was separated by silica gel column chromatography (development solvent: toluene/hexane), and the solid thus obtained was recrystallized from toluene/ethanol to produce 6.0 g of Compound 1 as a pale yellow crystalline solid (Yield 68%). The molecular weight of the product was measured by FAB-MS to be 806, the chemical formula thereof was taken to be C₆₀H₄₃N₃, and the target product was confirmed as Compound 1.

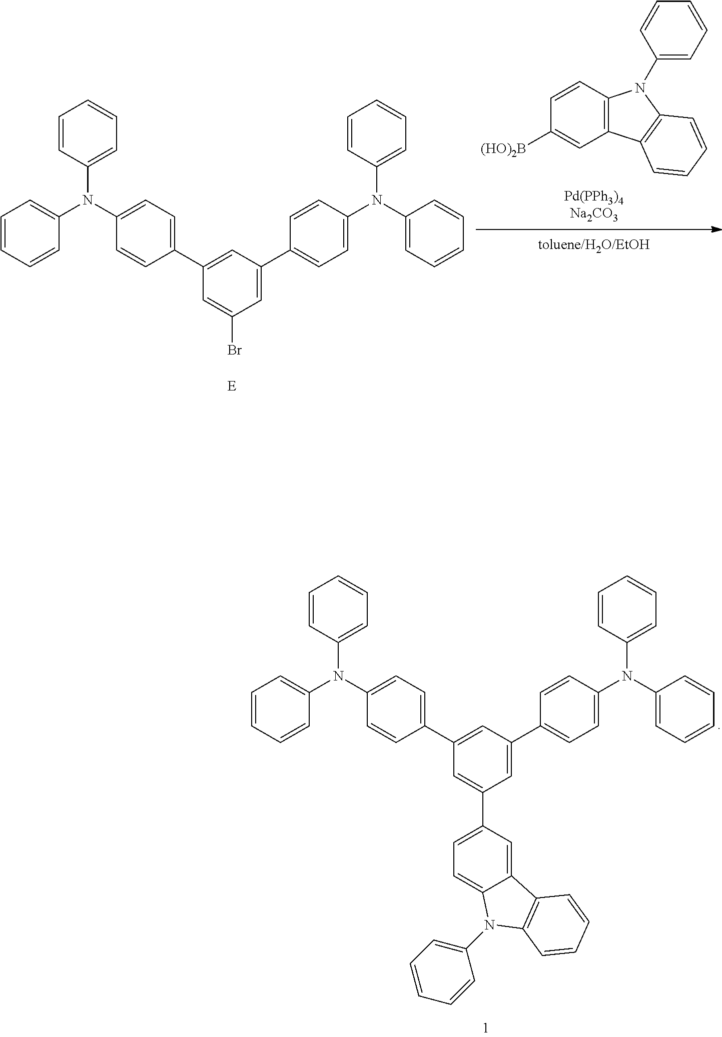 [SMILES: Brc1cc(-c2ccc(N(c3ccccc3)c3ccccc3)cc2)cc(-c2ccc(N(c3ccccc3)c3ccccc3)cc2)c1 ; OB(O)c1ccc2c(c1)c1ccccc1n2-c1ccccc1 ; c1ccc(N(c2ccccc2)c2ccc(-c3cc(-c4ccc(N(c5ccccc5)c5ccccc5)cc4)cc(-c4ccc5c(c4)c4ccccc4n5-c4ccccc4)c3)cc2)cc1]

Organic EL devices of Examples 1 to 7 were manufactured by respectively using Compounds 1 to 7 as hole transport materials, according to the manufacturing methods described above.

In addition, organic EL devices of Comparative Examples 1 to 3 were manufactured by respectively using the following Comparative Compounds C1 to C3 as hole transport materials.

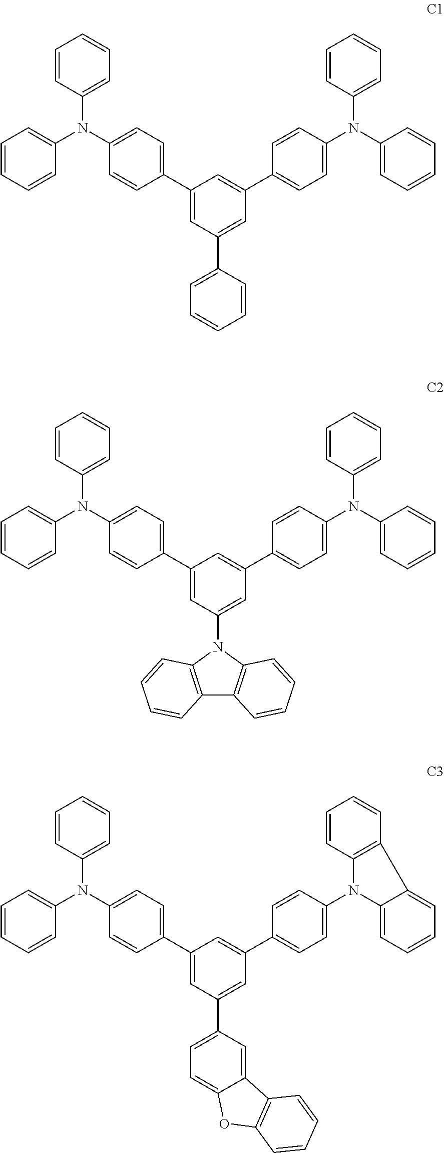 [SMILES: c1ccc(-c2cc(-c3ccc(N(c4ccccc4)c4ccccc4)cc3)cc(-c3ccc(N(c4ccccc4)c4ccccc4)cc3)c2)cc1 ; c1ccc(N(c2ccccc2)c2ccc(-c3cc(-c4ccc(N(c5ccccc5)c5ccccc5)cc4)cc(-n4c5ccccc5c5ccccc54)c3)cc2)cc1 ; c1ccc(N(c2ccccc2)c2ccc(-c3cc(-c4ccc(-n5c6ccccc6c6ccccc65)cc4)cc(-c4ccc5oc6ccccc6c5c4)c3)cc2)cc1]

Organic EL devices of Examples 1 to 7 may be each independently represented by the organic EL device 200 shown in FIG. 2. In the organic EL device 200, the substrate 202 was formed using a transparent glass substrate, the anode 204 was formed using ITO to a thickness of about 150 nm, the hole injection layer 206 was formed using 2-TNATA to a thickness of about 60 nm, the hole transport layer 208 was formed to a thickness of about 30 nm using the respective hole transport materials, the emission layer 210 was formed using ADN doped with 3% TBP to a thickness of about 25 nm, the electron transport layer 212 was formed using Alq₃ to a thickness of about 25 nm, the electron injection layer 214 was formed using LiF to a thickness of about 1 nm, and the cathode 216 was formed using Al to a thickness of about 100 nm. Organic EL devices of and Comparative Examples 1 to 3 were manufactured according to substantially the same method as the organic EL devices of Examples 1 to 7.

Emission efficiencies were evaluated for the organic EL devices of Examples 1 to 7 and Comparative Examples 1 to 3. Emission efficiency values were measured at a current density of about 10 mA/cm². The evaluation results are shown in the following Table 1. The evaluation of the emission properties of the organic EL devices thus manufactured was conducted using a C9920-11 brightness light distribution characteristics measurement system, made by Hamamatsu Photonics Co.

TABLE 1

| Device manufactur- | Hole transport | Emission efficiency |
|---|---|---|
| ing example | material | (cd/A) |
| Example 1 | Compound 1 | 7.1 |
| Example 2 | Compound 2 | 6.8 |
| Example 3 | Compound 3 | 6.9 |
| Example 4 | Compound 4 | 6.8 |
| Example 5 | Compound 5 | 6.8 |
| Example 6 | Compound 6 | 6.4 |
| Example 7 | Compound 7 | 6.3 |
| Comparative | Comparative | 5.5 |
| Example 1 | Compound C1 | |
| Comparative | Comparative | 5.1 |
| Example 2 | Compound C2 | |
| Comparative | Comparative | 4.0 |
| Example 3 | Compound C3 | |

Referring to the results shown in Table 1, the organic EL devices of Examples 1 to 7 showed higher emission efficiencies than the organic EL devices of Comparative Examples 1 to 3. Without being bound by any particular theory, it is believed that these results are at least in part due to the fact that in the material for an organic EL device according to embodiments of the present disclosure, two amine parts (e.g., amine moieties) are combined (e.g., coupled) via a 1,3,5-trisubstituted benzene moiety at positions 1 and 3 of the trisubstituted benzene ring. Thus, conjugation in the molecule may not increase (e.g., conjugation in the molecule may be limited), the energy gap may increase, and the emission efficiency of the organic EL device may be improved. In addition, the introduction of a heteroaryl group to the 5-position of the trisubstituted benzene may further improve the charge transport properties of a layer including the diamine compound according to embodiments of the present disclosure, and emission efficiency of the organic EL device may be improved. In contrast, in Comparative Example 1, the third substituent on the trisubstituted benzene is an aryl group, and thus the emission efficiency of the organic EL device of Comparative Example 1 is deteriorated when compared to those in Examples 1 to 7. In Comparative Example 2, the third substituent on the trisubstituted benzene is a carbazolyl group bonded through the heteroatom (N), which may change the electron donating properties of the carbazolyl group and the charge state of the molecule; and a decrease in emission efficiency may be observed. In Comparative Example 3, one of the two amine parts (e.g., amine moieties) combined (e.g., coupled) via the trisubstituted benzene at positions 1 and 3 of the trisubstituted benzene ring is replaced with a carbazolyl group, which may change the charge transport properties; and the emission efficiency may be deteriorated.

From the results shown in Table 1, it may be recognized that the organic EL devices using the material for an organic EL device according to embodiments of the present disclosure as a hole transport material, show higher efficiencies than those using the compounds of the comparative examples. In the material for an organic EL device according to embodiments of the present disclosure, two amine parts (e.g., amine moieties) are combined (e.g., coupled) via a 1,3,5-trisubstituted benzene at positions 1 and 3 of the trisubstituted benzene ring. Thus, conjugation in the molecule may not increase (e.g., may be limited), the energy gap may increase, and a high emission efficiency may be achieved in the corresponding organic EL device. The inclusion of a heteroaryl group to the 5-position of the trisubstituted benzene may further improve the charge transport properties and the high emission efficiency of the organic EL device may be realized.

In the material for an organic EL device according to embodiments of the present disclosure, since the two amine parts (e.g., amine moieties) are combined (e.g., coupled) via a 1,3,5-trisubstituted benzene moiety at positions 1 and 3, conjugation in the molecule may not increase (e.g., may be limited), the energy gap may increase, and the emission efficiency of the organic EL device may be improved. In addition, the introduction of a heteroaryl group to the 5-position trisubstituted benzene may further improve the charge transport properties and the emission efficiency of the organic EL. In addition, since the material for an organic EL device in one or more embodiments of the present disclosure has a wide energy gap, it may be possible to apply the material in the green to red emission regions.

According to one or more embodiments of the present disclosure, a material for an organic EL device with a high emission efficiency and an organic EL device using the same may be achieved. According to one or more embodiments, the material for an organic EL device relates to a diamine compound in which two amine moieties are combined (e.g., coupled) via a 1,3,5-trisubstituted benzene moiety at positions 1 and 3 of the trisubstituted benzene ring. Since the two amine parts (e.g., amine moieties) are combined (e.g., coupled) via the trisubstituted benzene, conjugation in the molecule may not be wide (e.g., conjugation in the molecule may be limited), the energy gap may increase, and the emission efficiency of the organic EL device may be improved. The inclusion of a heteroaryl group in the 5-position of the trisubstituted benzene may further improve the charge transport properties and the emission efficiency of the organic EL device. For example, improved characteristics may be obtained in the blue emission region.

As used herein, expressions such as “at least one of,” “one of,” “at least one selected from,” and “one selected from,” when preceding a list of elements, modify the entire list of elements and do not modify the individual elements of the list. Further, the use of “may” when describing embodiments of the present invention refers to “one or more embodiments of the present invention”.

In addition, as used herein, the terms “use,” “using,” and “used” may be considered synonymous with the terms “utilize,” “utilizing,” and “utilized,” respectively.

As used herein, the terms “substantially,” “about,” and similar terms are used as terms of approximation and not as terms of degree, and are intended to account for the inherent deviations in measured or calculated values that would be recognized by those of ordinary skill in the art.

Also, any numerical range recited herein is intended to include all subranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein. All such ranges are intended to be inherently described in this specification such that amending to expressly recite any such subranges would comply with the requirements of 35 U.S.C. §112(a) and 35 U.S.C. §132(a).

The above-disclosed subject matter is to be considered illustrative and not restrictive, and the appended claims and equivalents thereof are intended to cover all such modifications, enhancements, and other embodiments, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A material for an organic electroluminescent (EL) device, the material represented by Formula 1: 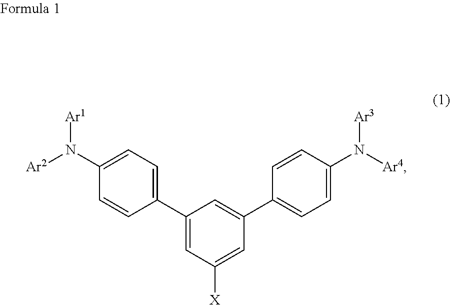
wherein in Formula 1,
Ar₁ to Ar₄ are each independently selected from a substituted or unsubstituted aryl group having 6 to 30 carbon atoms for forming a ring and a substituted or unsubstituted heteroaryl group having 5 to 30 carbon atoms for forming a ring, and
X is a substituted or unsubstituted heteroaryl group represented by Formula 2: 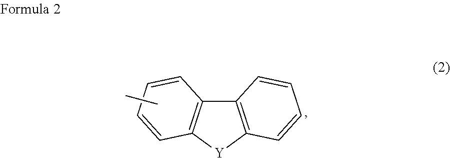
wherein in Formula 2,
Y is selected from O, S and NR, and
R is selected from a substituted or unsubstituted aryl group having 6 to 30 carbon atoms for forming a ring and a substituted or unsubstituted heteroaryl group having 5 to 30 carbon atoms for forming a ring.

2. The material for an organic EL device of claim 1, wherein Ar₁ to Ar₄ are each independently selected from a phenyl group, a biphenyl group, a naphthyl group, a terphenyl group and a phenanthryl group.

3. The material for an organic EL device of claim 1, wherein the heteroaryl group represented by Formula 2 is a monovalent group selected from the group consisting of a 1-carbazolyl group, a 2-carbazolyl group, a 3-carbazolyl group, a 4-carbazolyl group, a 1-dibenzofuranyl group, a 2-dibenzofuranyl group, a 3-dibenzofuranyl group, a 4-dibenzofuranyl group, a 1-dibenzothiophenyl group, a 2-dibenzothiophenyl group, a 3-dibenzothiophenyl group, a 4-dibenzothiophenyl group, and mixtures thereof.

4. The material for an organic EL device of claim 1, wherein the material for an organic EL device represented by Formula 1 is selected from one of the following Compounds 1 to 7:

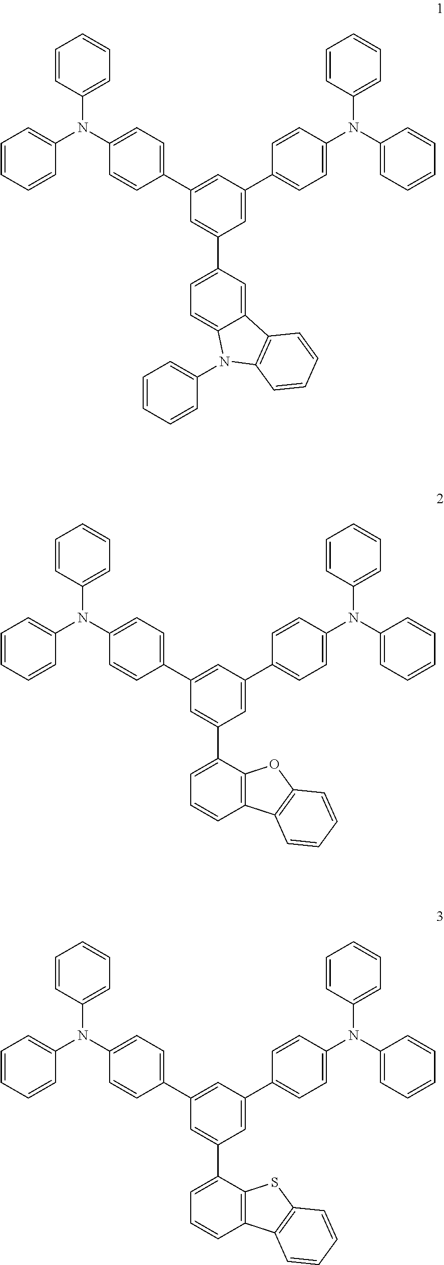 [SMILES: c1ccc(N(c2ccccc2)c2ccc(-c3cc(-c4ccc(N(c5ccccc5)c5ccccc5)cc4)cc(-c4ccc5c(c4)c4ccccc4n5-c4ccccc4)c3)cc2)cc1 ; c1ccc(N(c2ccccc2)c2ccc(-c3cc(-c4ccc(N(c5ccccc5)c5ccccc5)cc4)cc(-c4cccc5c4oc4ccccc45)c3)cc2)cc1 ; c1ccc(N(c2ccccc2)c2ccc(-c3cc(-c4ccc(N(c5ccccc5)c5ccccc5)cc4)cc(-c4cccc5c4sc4ccccc45)c3)cc2)cc1]

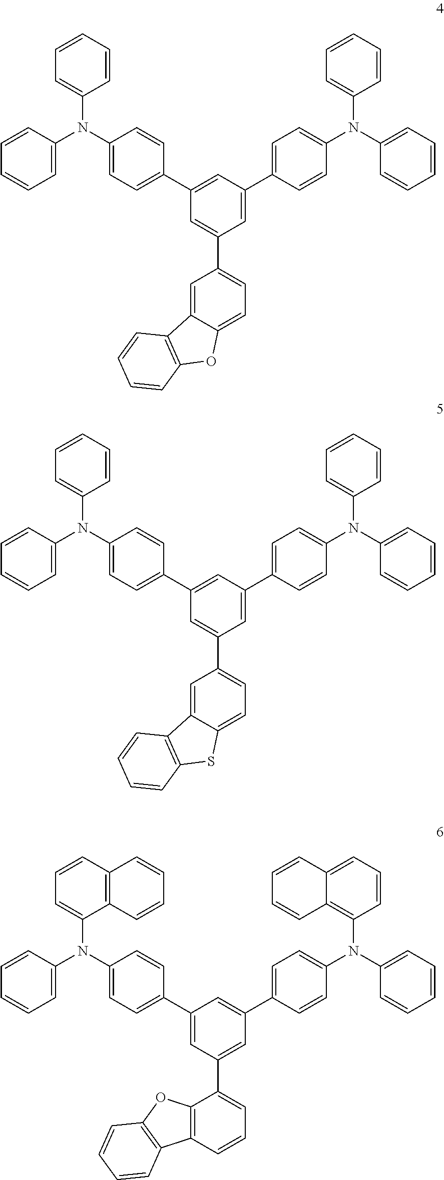 [SMILES: c1ccc(N(c2ccccc2)c2ccc(-c3cc(-c4ccc(N(c5ccccc5)c5ccccc5)cc4)cc(-c4ccc5oc6ccccc6c5c4)c3)cc2)cc1 ; c1ccc(N(c2ccccc2)c2ccc(-c3cc(-c4ccc(N(c5ccccc5)c5ccccc5)cc4)cc(-c4ccc5sc6ccccc6c5c4)c3)cc2)cc1 ; c1ccc(N(c2ccc(-c3cc(-c4ccc(N(c5ccccc5)c5cccc6ccccc56)cc4)cc(-c4cccc5c4oc4ccccc45)c3)cc2)c2cccc3ccccc23)cc1]

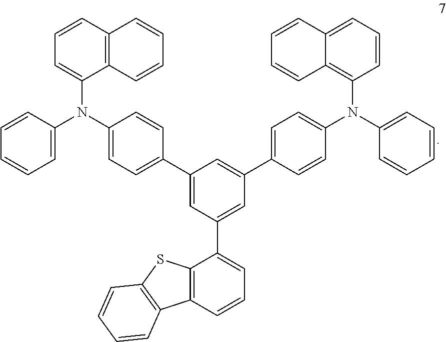 [SMILES: c1ccc(N(c2ccc(-c3cc(-c4ccc(N(c5ccccc5)c5cccc6ccccc56)cc4)cc(-c4cccc5c4sc4ccccc45)c3)cc2)c2cccc3ccccc23)cc1]

5. An organic electroluminescent (EL) device comprising an anode; a cathode facing the anode; and a plurality of layers between the anode and the cathode,
wherein at least one layer selected from the plurality of layers comprises a material for an organic EL device represented by Formula 1: 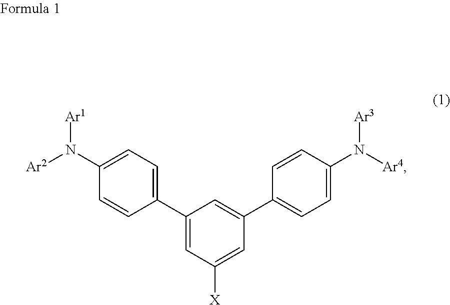
wherein in Formula 1,
Ar₁ to Ar₄ are each independently selected from a substituted or unsubstituted aryl group having 6 to 30 carbon atoms for forming a ring and a substituted or unsubstituted heteroaryl group having 5 to 30 carbon atoms for forming a ring, and
X is a substituted or unsubstituted heteroaryl group represented by Formula 2: 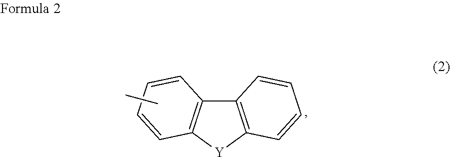
wherein in Formula 2,
Y is selected from O, S and NR, and
R is selected from a substituted or unsubstituted aryl group having 6 to 30 carbon atoms for forming a ring and a substituted or unsubstituted heteroaryl group having 5 to 30 carbon atoms for forming a ring.

6. The organic EL device of claim 5, wherein the plurality of layers between the anode and the cathode comprises an emission layer, and at least one layer between the anode and the emission layer comprises the material for an organic EL device.

7. The organic EL device of claim 5, wherein the plurality of layers between the anode and the cathode comprises an emission layer, and the emission layer comprises the material for an organic EL device.

8. The organic EL device of claim 6, wherein a hole transport layer is between the anode and the emission layer, the hole transport layer comprising the material for an organic EL device.

9. The organic EL device of claim 5, wherein Ar₁ to Ar₄ are each independently selected from a phenyl group, a biphenyl group, a naphthyl group, a terphenyl group and a phenanthryl group.

10. The organic EL device of claim 5, wherein the material for an organic EL device is selected from one of the following Compounds 1 to 7: 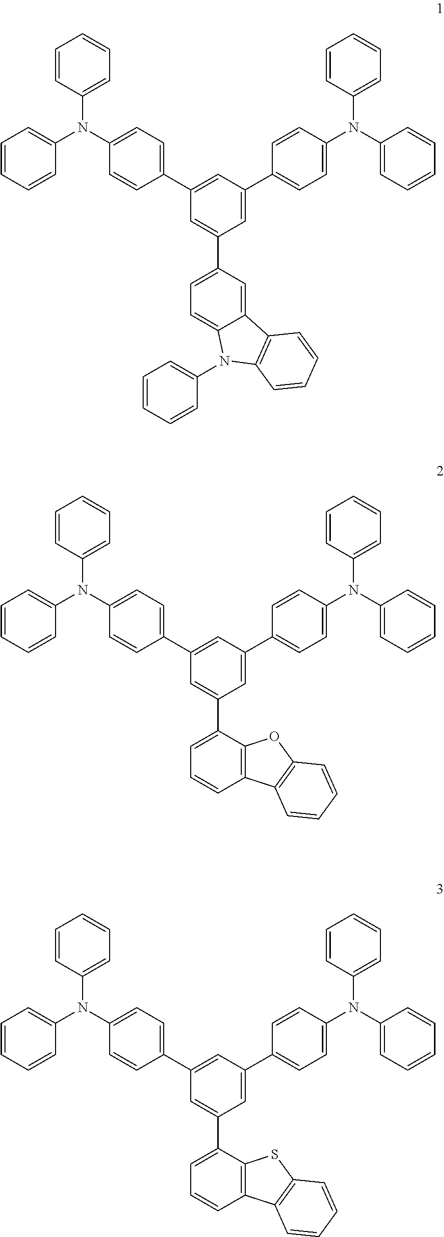 [SMILES: c1ccc(N(c2ccccc2)c2ccc(-c3cc(-c4ccc(N(c5ccccc5)c5ccccc5)cc4)cc(-c4ccc5c(c4)c4ccccc4n5-c4ccccc4)c3)cc2)cc1 ; c1ccc(N(c2ccccc2)c2ccc(-c3cc(-c4ccc(N(c5ccccc5)c5ccccc5)cc4)cc(-c4cccc5c4oc4ccccc45)c3)cc2)cc1 ; c1ccc(N(c2ccccc2)c2ccc(-c3cc(-c4ccc(N(c5ccccc5)c5ccccc5)cc4)cc(-c4cccc5c4sc4ccccc45)c3)cc2)cc1] 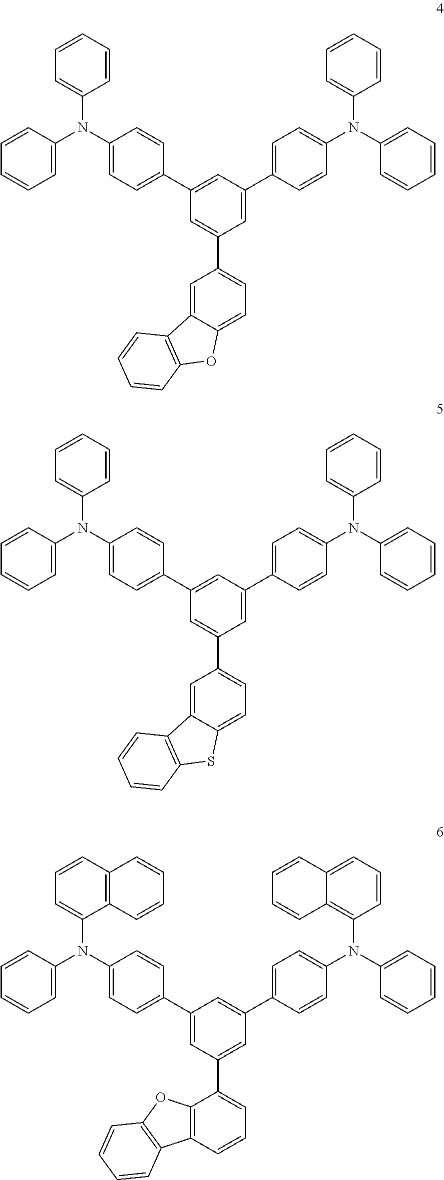 [SMILES: c1ccc(N(c2ccccc2)c2ccc(-c3cc(-c4ccc(N(c5ccccc5)c5ccccc5)cc4)cc(-c4ccc5oc6ccccc6c5c4)c3)cc2)cc1 ; c1ccc(N(c2ccccc2)c2ccc(-c3cc(-c4ccc(N(c5ccccc5)c5ccccc5)cc4)cc(-c4ccc5sc6ccccc6c5c4)c3)cc2)cc1 ; c1ccc(N(c2ccc(-c3cc(-c4ccc(N(c5ccccc5)c5cccc6ccccc56)cc4)cc(-c4cccc5c4oc4ccccc45)c3)cc2)c2cccc3ccccc23)cc1] 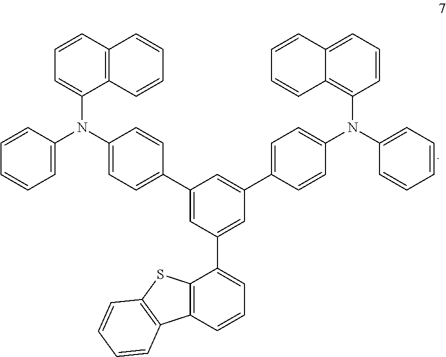 [SMILES: c1ccc(N(c2ccc(-c3cc(-c4ccc(N(c5ccccc5)c5cccc6ccccc56)cc4)cc(-c4cccc5c4sc4ccccc45)c3)cc2)c2cccc3ccccc23)cc1]
